(19) United States
(12) Patent Application Publication
(10) Pub. No.: US 2010/0016931 A1
(43) Pub. Date: Jan. 21, 2010

(54) Method of Reducing Cholesterol Using Laser Energy

(75) Inventors: Steven C. Shanks, McKinney, TX (US); Ryan Maloney, Gilbert, AZ (US)

Correspondence Address: ETHERTON LAW GROUP, LLC, 2010 E. University Drive, Suite 25, Tempe, AZ, 85281, US

(73) Assignee: Therapy Products, Inc., McKinney, TX (US)

(21) Appl. No.: 12/554,673

(22) Filed: Sep. 4, 2009

Related U.S. Application Data
(63) Continuation-in-part of application No. 11/409,408, filed on Apr. 20, 2006.
Application No. 11/409,408 is a continuation-in-part of application No. 10/772,738, filed on Feb. 4, 2004, now Pat. No. 7,118,588.
Application No. 10/772,738 is a continuation-in-part of application No. 10/976,581, filed on Oct. 29, 2004.
Application No. 10/976,581 is a continuation-in-part of application No. 09/932,907, filed on Aug. 20, 2001, now Pat. No. 6,746,473.
(60) Provisional application No. 60/273,282, filed on Mar. 2, 2001.

(51) Int. Cl. A61N 5/06; A61B 19/00

(52) U.S. Cl. 607/89; 128/898

(57) ABSTRACT

The present invention is a treatment for reducing serum cholesterol levels. The method involves analyzing a patient's lipid levels to determine a therapeutically sufficient amount of laser energy to apply to reduce a patient's LDL level while preserving his or her HDL level. The therapeutically sufficient amount of laser energy is determined by conducting a standard blood draw from the patient, conducting a lipid panel, and analyzing the results. In the preferred embodiment, a laser having a wavelength of about 635 nm is used to apply a therapeutic amount of laser energy of approximately 6.6 J/cm², provided over six treatments, each laser treatment two days apart.

### CROSS-REFERENCE TO RELATED APPLICATIONS

This application is a continuation-in-part of and claims the benefit of U.S. patent application Ser. No. 11/409,408 filed Apr. 20, 2006, which claims the benefit of co-pending U.S. patent application Ser. Nos. 10/772,738 filed Feb. 4, 2004, and U.S. patent application Ser. No. 10/976,581 filed Oct. 29, 2004, both of which claim the benefit of U.S. patent application Ser. No. 09/932,907 filed Aug. 20, 2001, now U.S. Pat. No. 6,746,473, which claims the benefit of U.S. Provisional Application No. 60/273,282 filed Mar. 2, 2001.

### FIELD OF INVENTION

This invention relates generally to methods for reducing cholesterol. This invention relates particularly to reducing cholesterol levels with the non-invasive application of laser energy.

### BACKGROUND

Nonfamilial hypercholesterolemia is defined by the American Heart Association as a serum cholesterol concentration exceeding 200 mg/dL. Nonfamilial hypercholesterolemia is the most common form of elevated serum cholesterol concentrations. The primary etiology for an overwhelming majority of patients is caused by an unknown genotype and further provoked by the excessive intake of saturated fat, trans-fatty acids, and cholesterol. Lipoprotein subpopulations levels, including those of low-density lipoproteins (LDL), very low-density lipoproteins (VLDL), triglycerides, and high-density lipoproteins (HDL), are evaluated to assess patient risk.

In the United States, 100 million people have serum cholesterol levels greater than 200 mg/dL, with 34.5 million people having levels exceeding 240 mg/dL. Several epidemiological studies have demonstrated a relationship between increased serum cholesterol concentrations and coronary heart disease events and related mortality rates. Moreover, elevated cholesterol levels have been linked to comorbidities such as atherosclerosis, stroke, and myocardial infarction.

The treatment regimen for patients with hypercholesterolemia is based on LDL, HDL, and triglyceride levels as modified by risk factors and history of previous CHD or risk equivalents including cigarette smoking, hypertension, age, and previous Ml or stroke. Current medical treatment involves measuring the lipids and fatty substances in the body by means of a blood test. Lipid panels usually include measurement of total cholesterol, triglycerides, HDL, and LDL. Other measurements that may be done for a lipid panel include that of VLDL, the ratio of total cholesterol to HDL, and the ratio of LDL to HDL.

Current medical treatment involves drug therapy in addition to lifestyle modifications. With the majority of cholesterol within the body arising via biosynthesis, clinicians aim to reduce cholesterol levels by prescribing hydroxyl methylglutaryl-CoA (HMG-CoA) reductase inhibitors, also referred to as statins, to inhibit the enzyme responsible for cholesterol synthesis regulation. Statins are competitive inhibitors of HMG-CoA reductase, therefore effectively inhibiting the enzyme by blocking the activation site. Statins decrease synthesis of cholesterol by the liver and other tissues. This action in liver decreases availability of cholesterol for the synthesis of VLDL particles that eventually become LDL particles.

Statins have become one of the most widely utilized class of medication in the United States. Although the more serious side-effects are rare, the risk of mild and even severe events can occur when taking statins. Approximately 5% of patients will report experiencing some muscle pain. In clinical trials, transaminase levels were elevated in patients taking statins, however serious liver toxicity is rare and remains a controversial topic amongst medical professionals. It would be desirable to reduce LDL levels without the side-effects caused by statins.

In recent years, the application of low-level laser light has garnered an exceptional level of interest across a myriad of medical disciplines because of its unique ability to modulate cellular metabolism thereby inducing beneficial clinical effects. Low-level laser therapy (LLLT) has been found to alter gene expression, cellular proliferation, intra-cellular pH balance, mitochondrial membrane potential, generation of transient reactive oxygen species and calcium ion level, proton gradient and cellular oxygen consumption. LLLT also improves wound healing, reduces edema, and relieves pain of various etiologies, including the treatment and repair of injured muscles and tendons.

LLLT utilizes low level laser energy, that is, the treatment has a dose rate that causes no immediate detectable temperature rise of the treated tissue and no macroscopically visible changes in tissue structure. Consequently, the treated and surrounding tissue is not heated and is not damaged. It would be desirable to use LLLT to serve as a non-pharmacological, non-invasive method to reduce serum cholesterol levels.

Therefore, it is an object of this invention to provide a method of reducing serum cholesterol levels. It is another object to this invention to provide a method of reducing serum cholesterol levels using low level laser therapy. It is a further object to provide a method that reduces serum cholesterol levels without drugs. It is a further object to provide a method that reduces serum cholesterol levels non-invasively.

### SUMMARY OF THE INVENTION

The present invention is a treatment for reducing serum cholesterol levels. The method involves determining a patient's body mass index (“BMI”) to determine a therapeutically sufficient amount of laser energy to apply to reduce the patient's LDL level while preserving his or her HDL level. The BMI and lipid levels are determined by methods known the art. In the preferred embodiment, a laser having a wavelength of about 635 nm is used to apply a therapeutic amount of laser energy of at least about 15 joules.

### BRIEF DESCRIPTION OF THE DRAWINGS

FIG. 1 is a flow diagram of the method of the present invention.

FIG. 2 is a side view of a patient being treated by a multi-laser scanner device using the method of the present invention.

FIG. 3 is a side view of a patient being treated by a hand-held laser scanner device using the method of the present invention

FIG. 4 illustrates a portable, floor-supported version of the laser device used in the preferred embodiment of the present invention.

FIG. 5 illustrates a wall-mounted version of the laser device used in the preferred embodiment of the present invention.

FIG. 6 is a schematic illustration of a laser device used in the preferred embodiment of the present invention.

FIG. 7 is a perspective view of a laser device used in the preferred embodiment of the present invention.

FIG. 8 is a perspective view of a scanning head of laser device used in the preferred embodiment, exploded along axes a and b.

FIG. 9 is a universal carriage holding a prism.

FIG. 9a is an exploded view of a prism holder and a prism.

FIG. 10 is a top-view of a multi-laser scanner.

FIG. 11 is the adults' body mass index table.

FIG. 12 is the children's body mass index table.

FIG. 13 illustrates scan patterns of laser energy application.

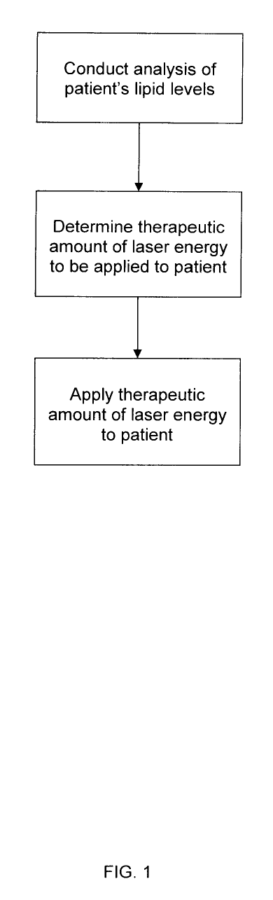

### DETAILED DESCRIPTION OF THE INVENTION

The amount of laser energy necessary to reduce serum cholesterol levels differs from person to person, depending on a variety of factors including BMI, density of fat irradiated, total cholesterol, triglycerides, HDL, and LDL. In the simplest embodiment of this invention, the amount of laser energy administered for LDL reduction depends only on BMI.

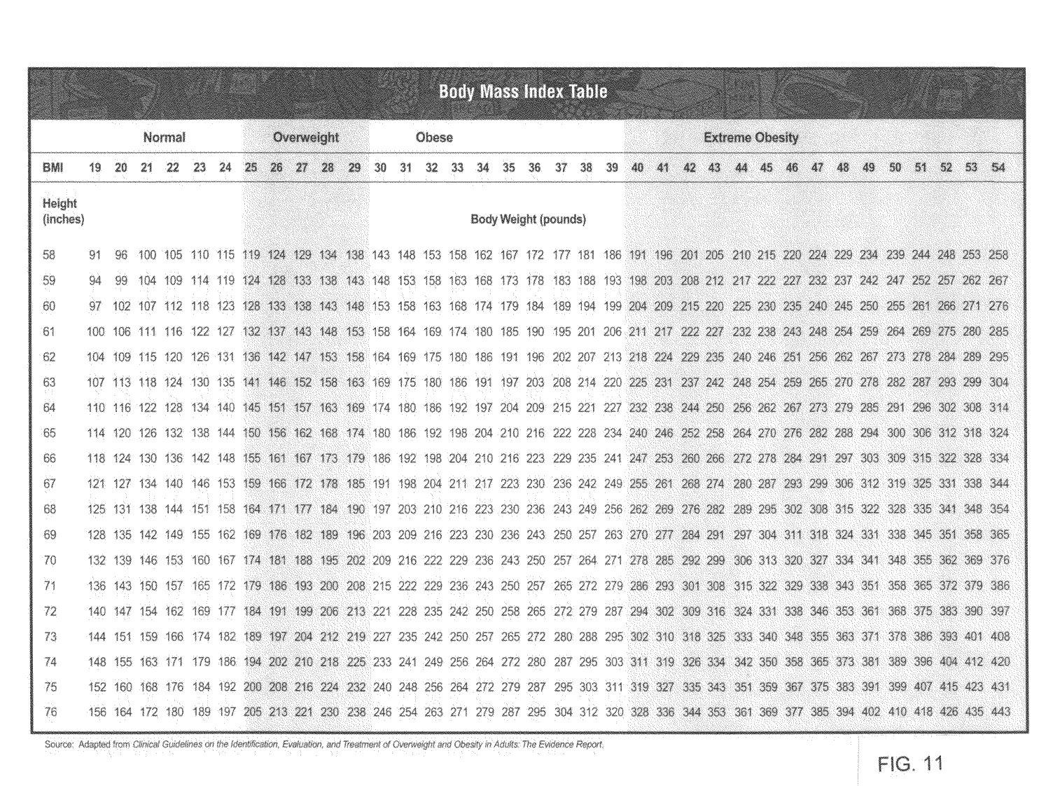

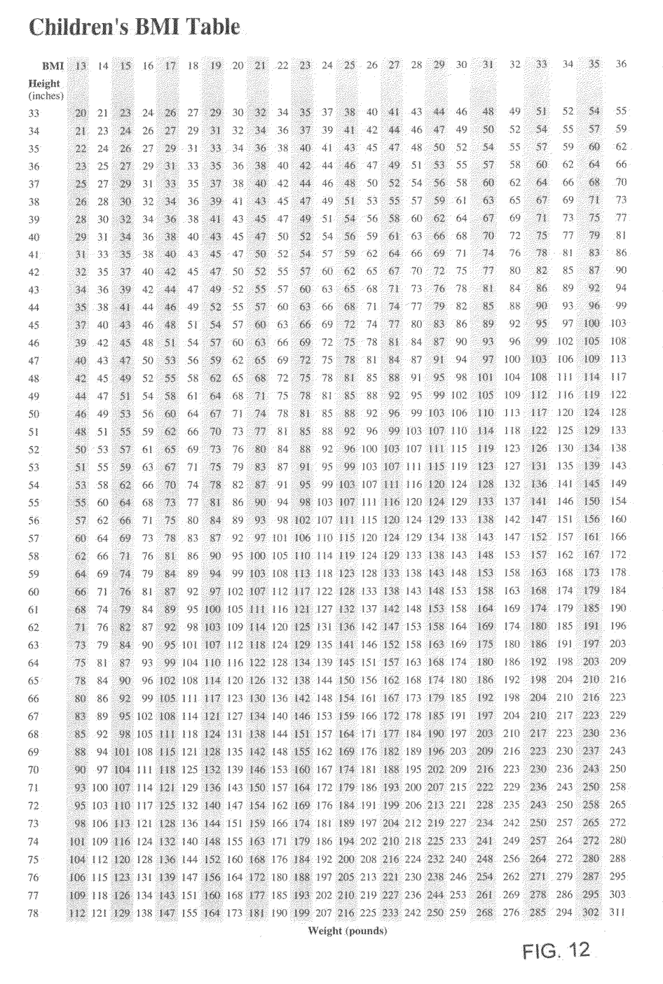

BMI is calculated by methods known the art. The preferred method involves measuring a patient's height and weight then looking up the BMI on the BMI tables promulgated by the NIH. FIGS. 11 and 12 are the adults' and children's, respectively, BMI tables adapted from the Clinical Guidelines of the Identification, Evaluation and Treatment of Overweight and Obesity in Adults: The Evidence Report, as reported by the National Heart Lung and Blood Institute of the National Institutes of Health.

To determine the therapeutic amount of laser energy that is sufficient to reduce the LDL level, the patient's BMI is determined. If the BMI is 15-24, at least about 15 joules of laser energy is applied to the patient. If the BMI is 25-31, about 21 joules of laser energy is applied to the patient. If the BMI is above 31, about 30 joules of laser energy is applied to the patient. See Table 1. Preferably the laser energy is applied to an area on the patient of about 400 cm², however multiple areas can be treated at the same time to reduce overall treatment time.

TABLE 1

| BMI | Laser Energy Applied (joules) |
|---|---|
| 15-24 | 15 |
| 25-31 | 21 |
| above 31 | 30 |

Further refinement of the amount of laser energy sufficient to reduce the LDL level, and the extent of additional treatments needed, can be made by measuring total cholesterol, triglycerides, HDL, and LDL before and after each laser treatment. Lipid measurements are made by conducting a standard blood draw and running a lipid panel, as known in the art. Other measurements that may be valuable for determining the amount of laser energy sufficient to reduce LDL include VLDL cholesterol level, the ratio of total cholesterol to HDL, and the ratio of LDL to HDL.

The laser energy can be applied to the patient using a variety of laser devices, such as a hand-held laser device, a wall-mounted laser device, or a stand-alone laser device, as described in more detail below. Preferably the laser device is a multi-diode laser scanner, also described in more detail below.

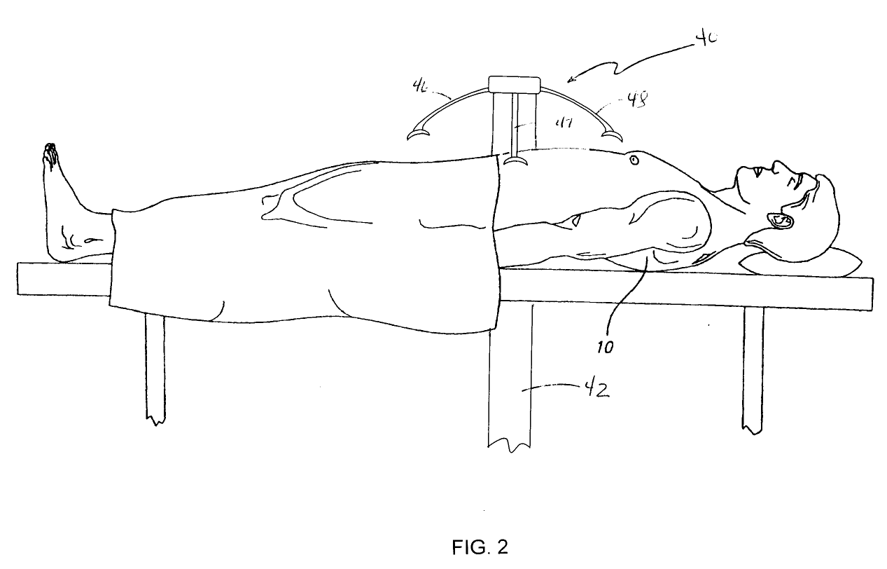

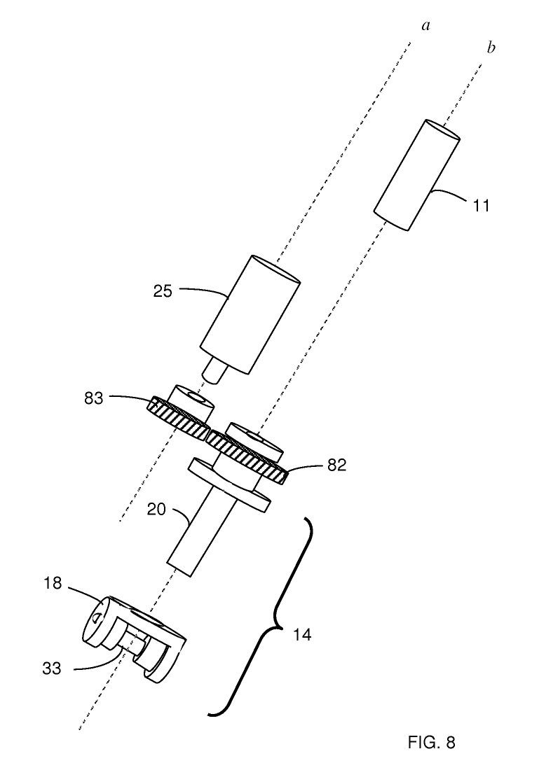

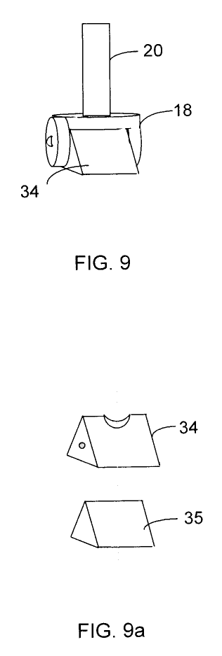

The laser energy is applied to the patient, preferably without the laser touching the patient. The laser energy can be applied to any location on the patient, but in the preferred embodiment, the therapeutic amount of laser energy is applied as follows. See FIG. 2. The patient 10 lies comfortably on his or her back. The center diode of a laser device 40, as described in more detail below and shown in FIG. 8, is positioned at a distance of about 6 inches above the patient's abdomen, centered along the body's midline and focused on the navel. The four remaining diodes are positioned 120 degrees apart and tilted 30 degrees off the centerline of the center diode. The laser device is activated for 20 minutes. Following anterior stimulation, the patient 10 turns over and lies on his or her stomach. The center diode of the laser device 40 is positioned at a distance of about 6 inches above the patient's back, centered along the body's midline and focused on the equivalent spot to the navel's location on the stomach. The four remaining diodes are again positioned 120 degrees apart and tilted 30 degrees off the centerline of the center diode. The laser device is activated again for 20 minutes.

For anterior and posterior treatments, the laser energy applied uses about 635 nm (red) wavelength. Preferably the laser energy is generated by one or more semiconductor laser diodes and generate about 17 mW output each. The patient is treated for a time sufficient to deliver at least 15 joules; more laser energy may be necessary for patients of higher BMI, higher fat density, or higher LDL levels.

Most low level laser treatments have proven to be effective at a single wavelength in the red region of the spectrum, between about 630 nm and about 670 nm. However, it has been shown that LLLT can be effective throughout the visible, near infrared and near ultraviolet regions. Laser diodes are currently available to cover only a limited part of the available spectrum, so other laser energy sources may be used. To obtain maximum benefit it may be desirable to stimulate the patient at two or more different wavelengths. Persons skilled in the art will be aware that various laser energy sources are known in the art for use in low-level laser therapy. They include Helium-Neon lasers having a 632 nm wavelength and semiconductor diode lasers with a broad range of wavelengths between 600-800 nm. The laser energy source in the preferred embodiment is a semiconductor laser diode that produces light in the red range of the visible spectrum, having a wavelength of about 635 nm. Other suitable wavelengths are used for other particular applications. The preferred embodiment is described as having a single laser energy source 11 but it will be appreciated that the invention may have two or more laser energy sources. These laser sources may be attached to each other in a laser assembly or individually attached to a support structure. While many LLLT regimen include ultraviolet or infrared laser light, it is advantageous to utilize at least one laser beam in the visible energy spectrum so that the operator can see the laser light as it impinges the patient's body and the area treated can be easily defined.

Different therapy regimens require diodes of different wattages. The preferred laser diodes use less than one watt of power each to simultaneously facilitate liposuction, treat post-operative inflammation and post-operative pain, as well as to restore hair. Diodes of various other wattages may also be employed to achieve the desired laser energy for the given regimen. It may be advantageous to provide a power source separate from the housing, and deliver the power to the housing by wire. An advantage of the present invention is that a larger treatment area can be achieved without the need for a higher power laser.

Laser control 13, preferably incorporated into control means 17, controls the duration of each pulse of laser light emitted and the pulse frequency. When there are no pulses, a continuous beam of laser light is generated. Pulse frequencies from 0 to 100,000 Hz or more may be employed to achieve the desired effect on the patient's tissue. The goal for LLLT regimen is to deliver laser energy to the target tissue utilizing a pulse width short enough to sufficiently energize the targeted tissue and avoid thermal damage to adjacent tissue.

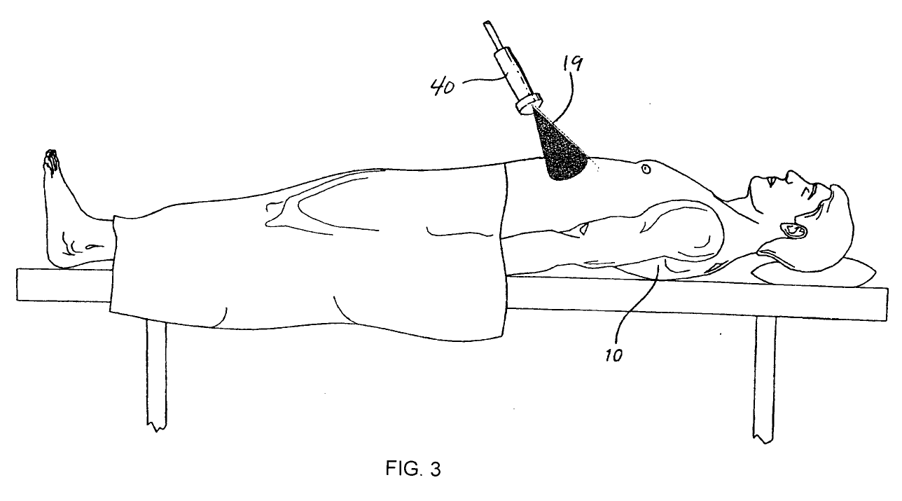

The laser energy can be applied with a hand-held laser device, as shown in FIG. 3 and described in detail in U.S. Pat. No. 6,746,473. The laser light may be directed to the desired area on a patient using a hand-held probe. The probe is a housing comprising an elongated hollow tube defining an interior cavity. In the preferred embodiment the laser energy source 11 is mounted in the housing's interior cavity, although the laser energy source could be remotely located and the laser light conducted by fiber optics to the housing. The housing may take on any shape that enables the laser light to be directed as needed such as tubular, T-shaped, substantially spherical, or rectangular. The housing may contain the power supply (for example a battery) or the power supply may be remote with power supplied by an electrical cable. A scanning head may be contained wholly within each housing or attached separately to the end of each housing.

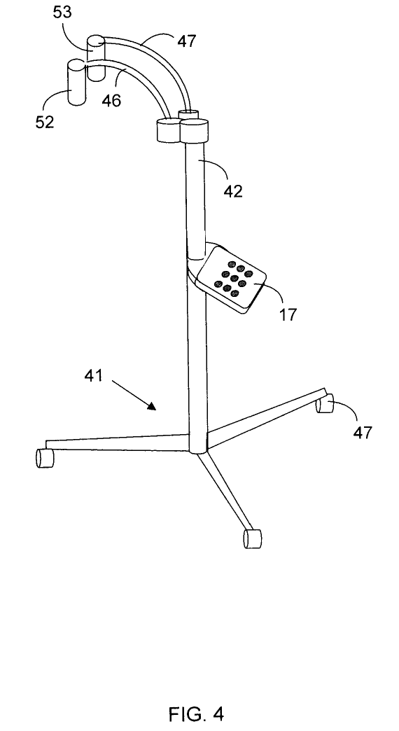

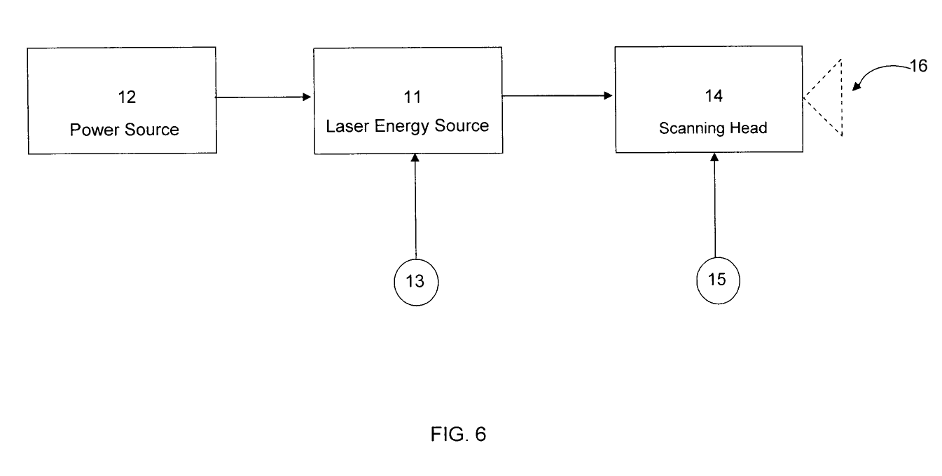

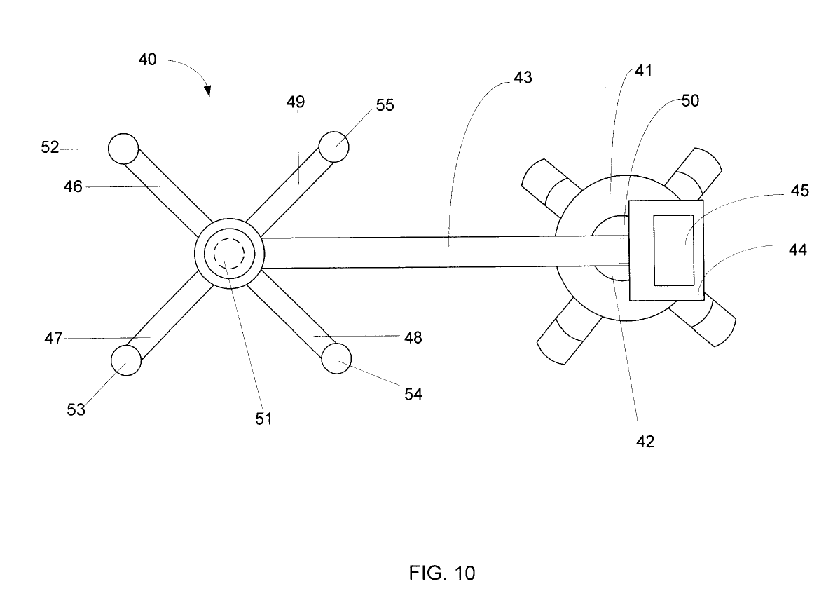

The laser device may also operate in a stand-alone configuration. For example, the present device may be supported by a support structure such as the wall or a portable stand that rests on the floor or table. This stand-alone arrangement enables a patient to be scanned by the laser beam without movement of the diode. FIG. 4 shows the portable, floor-mounted version of the present invention. Laser diodes 52 and 53 are attached to arm 43 with scanner arms 46 and 47, respectively. The scanner arms may be rigid or, preferably, flexible, so that the diodes can be moved to any desired position. The vertical support 42 and scanner arms 46 and 47 may be articulated for additional control over the position of the lasers. The vertical support 42 is attached to a base 41 having wheels 47 such that the device can be moved to any desired position and then stay substantially stationary while treatment is occurring. This is particularly convenient for patients lying on a table or sitting in wheelchair. Control means 17 is in electrical connection with the diodes and is shown in FIG. 6 mounted on the vertical support 42. The control, however, can be mounted elsewhere or can operate as a remote control using radio frequencies or other methods known in the art. FIGS. 2 and 10 show the preferred embodiment of a stand-alone laser device, a multi-laser scanning device having 5 independent laser diodes. These laser devices are sold under the trademarks LIPOLASER™ and ZERONA™, and are available from Therapy Products, Inc.

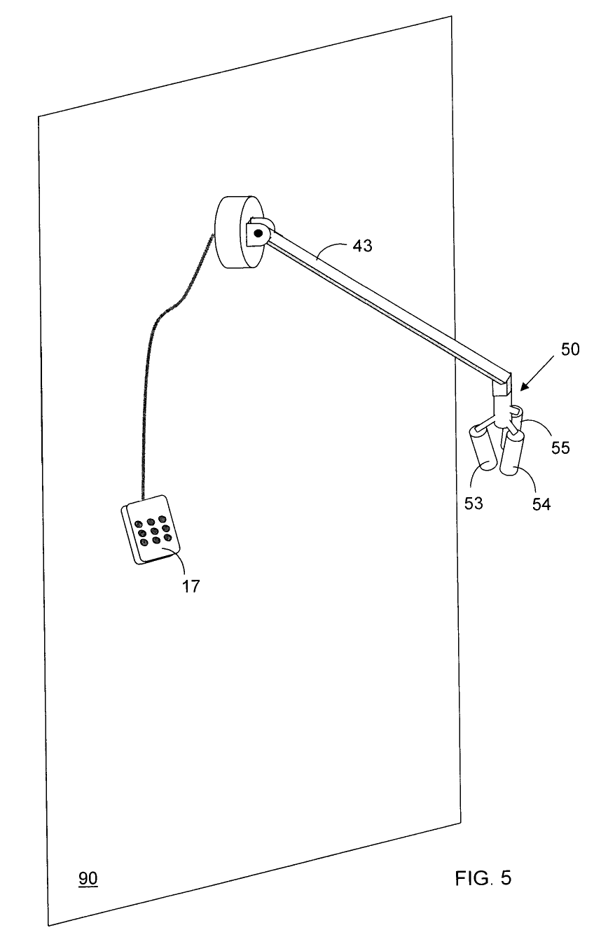

The laser energy can also be applied with a wall-mounted laser device, as shown in FIG. 5. FIG. 5 shows a three-diode assembly 50 attached to a wall-mounted arm 43. The arm 43 is affixed to the wall 90 in ways known in the art such that it can be moved to any desired position and then stay in substantially stationary while treatment is occurring. The arm may be articulated for additional control over the position of the lasers. Control means 17 is in electrical connection with the diodes and is shown in FIG. 5 mounted on the wall. The control, however, can be mounted elsewhere or can operate as a remote control using radio frequencies or other methods known in the art. The assembly 50 is attached to the arm 43 in ways known in the art such that it can be moved to any desired position. Likewise, the diodes 53, 54, 55 are attached to the assembly so that each can be moved to a desired position.

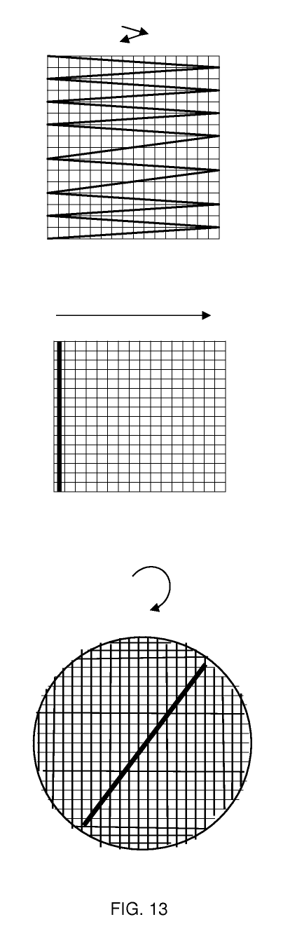

The laser energy can be administered various modalities. These modalities can be as simple as a hand held wand or probe waved over the treatment area to more complex configurations using an automatic or semiautomatic scanning device that moves the laser beam over the treatment area. Examples of scan patterns are shown in FIG. 13, where the direction of the laser scan is indicated by the arrow above each pattern.

Whether hand-held, floor-mounted or wall-mounted, preferably the laser device is a laser scanner. FIG. 6 illustrates a schematic of a laser device that includes a power source 12, at least one laser energy source 11, a laser control 13, a scanning head 14, and a scanner control 15. In the preferred embodiment, the laser control 13 and scanner control 15 are incorporated into a control means 17. The power source preferably provides direct current, such as that provided by a battery, but may instead provide alternating current such as that provided by conventional building outlet power (e.g. 120V) that is then converted to direct current. The power supply 12 may be housed with the scanning head 14 or may be deployed separately with an electrical cable joining it thereto. Laser control 13 is connected to the laser energy source 11 and acts as on/off switch to control the period of time the laser light is generated and may also have other functions, such as controlling the pulse frequency. Other functions of the laser control 13, scanner control 15, and control means 17 are mentioned below.

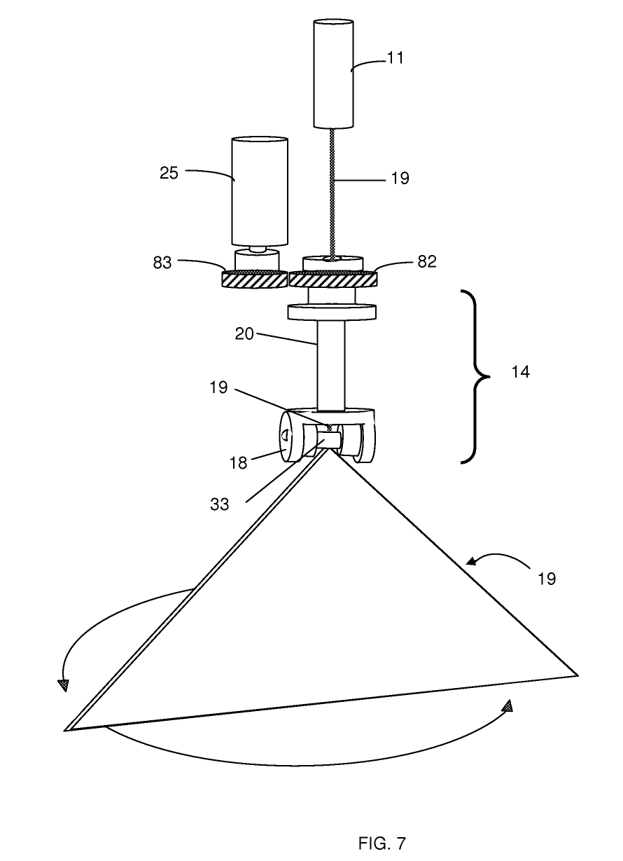

A laser beam 19 emitted from the laser source 11 is directed to the scanning head 14. See FIG. 7. In the preferred embodiment, the scanning head comprises a hollow spindle 20 through which the laser beam 19 is conveyed. A rotatable carriage 18 holds an optical element upon which the laser beam 19 is incident. In the preferred embodiment, the laser beam 19, spindle 20 and carriage 18 are substantially co-axial. See FIG. 3.

In the preferred embodiment, the optical element generates a line when laser light impinges on it. A rod lens 33 is preferred as the optical element, but a prism or other optical element or combination thereof may suffice. When the laser beam 19 strikes the optical element a line is generated. As the carriage 18 rotates, the line rotates, too, becoming, in essence, a rotating diameter of the apparent circular beam spot. If the carriage is rotated through 360°, the line also sweeps through a complete circle. See FIG. 7. With electronic or computerized control, the carriage is able to automatically rotate very quickly, causing the laser beam to appear to create a substantially circular beam spot on the patient's skin. The shape, however, is actually the result of the scanning light diameter sweeping from location to location at a speed that makes the motion nearly imperceptible to the human eye. The longer the line, the larger the beam spot.

The carriage is rotated with a drive assembly. The drive assembly is preferably a main drive gear 82 which is mated with a minor drive gear 83. The minor drive gear is driven by a main drive motor 25. The carriage 18 rotates around the axis as the main drive gear 82 is turned. Thus, the laser beam from laser energy source 11 passes through the hollow spindle 20 and strikes an optical element which deflects the laser beam into a line that, in combination with the rotation, appears as a circular beam spot. The drive assembly may also be controlled by micromanipulators according to signals received from the scanner control 15, which is preferably incorporated into control means 17. Preferably the control means 17 is further comprised of various discrete circuits, as is known in the art. In a further form, the control means 17 is a microprocessor programmed to operate in various modes.

The scanner control 15 may also be programmed to move the scanning head 14 in a required manner to achieve any desired path of a treatment zone on the skin of a patient. Furthermore, the scanner control 15 can be programmed to direct the laser output into some regions more than others so that one region may have greater treatment than another region. The scan areas may overlap. This may be particularly useful for stand-alone apparatuses using the present invention, for example in a stand-alone laser device. The invention is not limited to any particular programmed operation mode, but by way of example the following modes of operation are available:

1. The scanning head is programmed to move through a series of fixed regions and dwell for a pre-set period at each region. The regions may be input by a user to align with particular positions on the patient that require stimulation.

2. The wavelength is periodically changed by changing the operating laser diode during a repetitive scan. This allows stimulation of the patient at multiple wavelengths.

3. The focal position of the beam shaping optics is changed to generate smaller or larger spot sizes on the patient.

4. The laser power is varied.

The multi-diode laser device particularly useful for this application is the stand-alone multi-laser scanner shown in FIG. 10. This multi-laser scanner 40 is supported by a wheeled base 41. Preferably the wheeled base includes at least two locking wheels to ensure affixed placement. Additionally, the wheeled base preferably houses a charger jack for the unit and the mechanical components required to operate the multi-laser scanner. A height-adjustable vertical support 42 extends upwards from wheeled base 41. A freely rotatable boom arm 43 extends outward from vertical support 42. Preferably boom arm 43 is a two-jointed arm that offers placement flexibility with a bend off the base and before the head. Descending from the boom arm is the laser scanner unit which comprises four scanner arms 46, 47, 48, and 49. The scanner arms protrude from the center of the laser scanner unit and are flexible arms that allow for 90 degree rotation. At the center of the laser scanner unit is a first laser diode 51. Attached to each scanner arm are additional laser diodes 52, 53, 54 and 55. Each laser diode contains an independent diode of variable frequency and preferably having a 635 nm wavelength. The four heads 52, 53, 54, and 55 attached to scanner arms 46, 47, 48, and 49 are positioned 90 degrees apart from each other, and each is tilted at a 30 degree angle from the centerline of the center scanner. Each of the independent laser diodes is processed through a lens that redirects the beam with a line refractor. The refracted light is then bent into a spiraling circle pattern that is random and independent of the other diodes. These patterns overlap each other to provide coverage within the target area. Preferably each laser diode emits 17 mW, 635 nm of red laser light. Multi-laser scanner 40 also includes, in the preferred embodiment, a control center 44 and touch screen 45 that acts as the command module and the user's interface with the device. Additionally, a key lock (not shown) is positioned on multi-laser scanner 40 to lock the device. The unit is turned on with a key lock. Additional technical details of the multi-laser scanner are disclosed in US Patent Publication 2006/0095099.

A shield may be employed to prevent the laser light from reflecting or deflecting to undesired locations. The shield (not shown) is attached where appropriate to block the radiation. For example, the shield may be attached to the assembly, to one or more of the housings, or worn by the patient. The shield may take on a variety of shapes, as appropriate, depending on the area to be shielded. For example, the shield may take on a rectangular or hemi-cylindrical shape to shield a patient's upper torso.

In the preferred embodiment, each laser diode 52, 53, 54, 55 is directed by the control means 17 to deliver a desired scan pattern with a desired laser output across the patient. Due to the nature of the laser light, the patent can be treated without touching him or her with the laser. The laser diodes are preferably contained within housings.

While the preferred embodiment reduces serum cholesterol level without drugs, pharmacological treatment may be used in combination with the laser therapy.

While there has been illustrated and described what is at present considered to be a preferred embodiment of the present invention, it will be understood by those skilled in the art that various changes and modifications may be made, and equivalents may be substituted for elements thereof without departing from the true scope of the invention. Therefore, it is intended that this invention not be limited to the particular embodiment disclosed as the best mode contemplated for carrying out the invention, but that the invention will include all embodiments falling within the scope of the appended claims.

We claim:

1. A method of reducing low-density lipoprotein (“LDL”) level in a patient comprising:
a. determining the patient's body mass index (“BMI”);
b. determining from the BMI a therapeutic amount of laser energy that is sufficient to reduce the LDL level of the patient; and
c. applying the determined therapeutic amount of laser energy to the patient.

2. The method of claim 1 wherein the laser energy has a wavelength in the red range.

3. The method of claim 1 wherein the laser energy has a wavelength of about 635 nm.

4. The method of claim 1 wherein the determined therapeutic amount of laser energy is at least 15 joules.

5. The method of claim 1 wherein the BMI is 15-24 and the amount of laser energy applied is at least 15 joules.

6. The method of claim 1 wherein the BMI is 25-31 and the amount of laser energy applied is at least 21 joules.

7. The method of claim 1 wherein the BMI is higher than 31 and the amount of laser energy applied is at least 30 joules.

8. The method of claim 1 further comprising determining the patient's LDL level.

9. The method of claim 1 further comprising conducting a lipid panel and measuring at least LDL, HDL and total cholesterol.

10. The method of claim 9 wherein the LDL level indicates the amount of laser energy to be applied to the patient.

11. The method of claim 1 wherein the laser energy is applied by a laser device comprising:
a. at least one laser energy source for generating a laser beam;
b. means for causing the laser beam to rapidly scan; and
c. a support structure connected to laser energy source which enables a patient to be scanned by the laser beam without moving the laser energy source.

12. The method of claim 11 wherein the means for causing the laser beam to rapidly scan is a scanning head.

13. The method of claim 11 wherein the means for causing the laser beam to rapidly scan is a raster scanner.

14. A method of reducing serum cholesterol levels in a patient comprising:
a. drawing a first blood sample from the patient;
b. measuring the level of LDL in the first blood sample;
c. applying at least 15 joules of laser energy to the patient using a laser device;
d. drawing a second blood sample from a patient;
e. measuring the level of LDL in the second blood sample;
f. comparing the level of LDL in the first blood sample to the level of LDL in the second blood sample; and
g. if the level of LDL in the first blood sample is higher than the level of LDL in the second blood sample, applying additional laser energy to the patient using a laser device.

15. The method of claim 14 further comprising:
a. determining the patient's body mass index (“BMI”); and
b. determining from the BMI the additional amount of laser energy that is sufficient to reduce the LDL level in the first blood sample of the patient.

16. The method of claim 14 wherein the laser device further comprises a raster scanner to apply the laser energy.

17. The method of claim 14 wherein the laser device further comprises a scanning head to apply the laser energy.

18. The method of claim 14 wherein the laser device further comprises:
a. a laser energy source generating a laser beam;
b. a scanning head for receiving the laser beam and for directing the laser beam into a desired location, the scanning head comprising:
i. a pushrod having a tapered leading edge, a main drive gear and a rocker carriage, all aligned along a central axis;
ii. the rocker carriage comprising an optical element attached to a transverse axle;
iii. a spring clip biasing the optical element against the leading edge of the pushrod;
iv. a beveled cam abutting the main, the beveled can driven by a second drive motor to cause the pushrod to move up and down and thereby rotate the optical element;
v. a main drive gear connected to the rocker carriage; and
vi. a minor drive gear mated to the main drive gear and driven by a first drive motor to cause the rocker carriage to rotate about the central axis;
vii. a control circuit for controlling the scanning head;
such that the laser beam passes through the pushrod and strikes the optical element and forms a desired scan pattern.

19. The laser device according to claim 18 in which the optical element is a mirror.

20. The laser device according to claim 18 in which the optical element is a prism.
